(19) United States
(12) Patent Application Publication
(10) Pub. No.: US 2024/0000880 A1
(43) Pub. Date: Jan. 4, 2024

(54) A PHARMACEUTICAL COMPOSITION FOR PREVENTING AND TREATING LUNG INJURY, A PREPARATION METHOD THEREFOR AND A USE THEREOF

(71) Applicants: INSTITUTE OF MATERIA MEDICA , CHINESE ACADEMY OF MEDICAL SCICENCES, Beijing (CN); JILIN MAIFU NUTRITION TECHNOLOGY CO., LTD, Meihekou, Jilin (CN)

(72) Inventors: Hongtao JIN, Beijing (CN); Peicheng ZHANG, Beijing (CN); Jinlan ZHANG, Beijing (CN); Jiandong JIANG, Beijing (CN); Ping ZHANG, Beijing (CN); Yangyang LI, Beijing (CN)

(21) Appl. No.: 18/039,976

(22) PCT Filed: Dec. 3, 2021
(86) PCT No.: PCT/CN2021/135483

§ 371 Date: Jun. 2, 2023

(30) Foreign Application Priority Data
Dec. 4, 2020 (CN) 202011410183.6
May 12, 2021 (CN) 202110518014.2

(51) Int. Cl. A61K 36/87; A61K 9/14; A61K 31/05; A61K 31/198; A61K 31/375; A61K 31/355; A61K 47/42; A61K 45/06; A61P 11/00

(52) U.S. Cl. CPC A61K 36/87; A61K 9/14; A61K 31/05; A61K 31/198; A61K 31/375; A61K 31/355; A61K 47/42; A61K 45/06; A61P 11/00

(57) ABSTRACT

The disclosure relates to a pharmaceutical composition having the efficacy of preventing and treating lung injury, a preparation method therefor and a use thereof. The composition comprises a grape powder with a content of 1-25% (w/w), a grape seed extract with a content of 0.5-15% (w/w) and a pharmaceutically acceptable carrier. The composition of the disclosure has the efficacy of preventing and treating lung injury, oxidative injury, inhibiting pulmonary fibrosis, regulating lipid metabolism, and the like.

### FIELD

The disclosure relates to the field of biomedicine, in particular to a pharmaceutical composition having the efficacy of preventing and treating lung injury, a preparation method therefor and a use thereof.

### BACKGROUND

With the increasing air pollution, epidemiological research results show that fine particulate matter with a particle diameter less than 2.5 μm (PM2.5) seriously endangers human health, leads to respiratory diseases, and even involves organs such as cardiovascular system, nervous system, immune system. More and more attention has been paid to the impact of air pollution on the respiratory system, especially the correlation with chronic obstructive pulmonary disease (COPD). The intervention and prevention of COPD caused by PM2.5 have become a research hotspot.

Lung injury includes lung tissue injury caused by the factors such as chest trauma, lung inhalation of harmful substances and lung infection, and the conditions damaging the integrity of lung structure and lung function. Air pollutants are easily inhaled into the deep lung to cause respiratory tract injury, induce or aggravate a variety of diseases, and cause inflammation response, oxidative stress response, immune dysfunction and the like in the body. Lung injury is closely correlated with oxidative injury of tissue cells. Anti-inflammatory drugs, antioxidants and drugs regulating immune function are commonly used for prevention and treatment in clinical practice.

CN103099205A discloses a grape seed tablet including a grape seed extract, vitamin C and vitamin E, the grape seed tablet has efficacy of anti-oxidation, alleviating senile spots and reducing wrinkles, etc. CN103478561A discloses a functional food containing a grape seed extract, vitamin C and vitamin E, which has efficacy of anti-oxidation and anti-aging, etc.

Previous studies have shown that resveratrol has antioxidant efficacy on chronic obstructive pulmonary disease in rats (see Antioxidation and Its Mechanism of Resveratrol in Rats with Chronic Obstructive Pulmonary Disease, Journal of Kunming Medical University, 2013), and can improve lung function to some extent, but the efficacy is slow, and its efficacy on preventing and treating lung injury needs to be improved. Therefore, it is particularly pressing to research and develop pharmaceutical compositions for the prevention and treatment of lung injury.

### BRIEF SUMMARY

The purpose of the disclosure is to provide a pharmaceutical composition with efficacy of preventing and treating lung injury, the composition comprises a grape powder, a grape seed extract and a pharmaceutically acceptable carrier.

In the preferred embodiment of the disclosure, the content of the grape powder is of 1-25% (w/w) and the content of the grape seed extract is of 0.5-15% (w/w) in the composition.

In the preferred embodiment of the disclosure, the content of the grape powder is of 3-20% (w/w) and the content of the grape seed extract is of 2-12% (w/w) in the composition.

In the preferred embodiment of the disclosure, the content of the grape powder is of 6-15% (w/w) and the content of the grape seed extract is of 3-10% (w/w) in the composition.

In the preferred embodiment of the disclosure, the content of resveratrol in the grape powder is of 1-20% (w/w), preferably of 4-15% (w/w), more preferably of 5-8% (w/w).

In the preferred embodiment of the disclosure, the content of proanthocyanidins in the grape seed extract is of 90-100% (w/w), preferably of 95-98% (w/w).

In the preferred embodiment of the disclosure, the proanthocyanidins are oligomeric proanthocyanidins.

In the preferred embodiment of the disclosure, the pharmaceutically acceptable carrier is selected from fillers, antioxidants, flavoring agents or any combination thereof.

In the preferred embodiment of the disclosure, the content of the fillers in the composition is of 55-90% (w/w), preferably of 65-85% (w/w), more preferably of 70-80% (w/w).

In the preferred embodiment of the disclosure, the fillers are selected from maltodextrin, starch, lactose, sodium carboxymethyl starch, sodium starch glycolate, pregelatinized starch, modified starch, hydroxypropyl starch, corn starch, cellulose, microcrystalline cellulose, sodium carboxymethyl cellulose, ethyl cellulose, hydroxypropyl methylcellulose, calcium sulfate, calcium phosphate, calcium hydrogen phosphate, precipitated calcium carbonate, sorbitol, glycine, or any combination thereof.

In the preferred embodiment of the disclosure, the content of the antioxidants in the composition is of 0.5-15% (w/w), preferably of 1-12% (w/w), more preferably of 2-10% (w/w).

In the preferred embodiment of the disclosure, the antioxidants are selected from arginine, L-arginine, vitamin C, vitamin E, tert-butylhydroquinone, butyl hydroxyanisole, dibutyl hydroxytoluene, or any combination thereof.

In the preferred embodiment of the disclosure, the content of arginine, L-arginine, vitamin C, vitamin E or any combination thereof in the composition is of 0.5-15% (w/w), preferably of 1-12% (w/w), more preferably of 2-10% (w/w).

In the preferred embodiment of the present disclosure, the content of flavoring agents in the composition is 0.1-5% (w/w), preferably 0.2-4% (w/w).

In the preferred embodiment of the disclosure, the flavoring agents are selected from sucralose, grape essence, erythritol, xylitol, honey, sucrose, glucose, mogroside, malic acid, fumaric acid, citric acid, phosphoric acid, ethyl maltol, sodium citrate, sodium malate, acetic acid, sodium acetate, sodium hydrogen phosphate, sodium dihydrogen phosphate, carbonic acid, sodium carbonate, sulfonic acid, sodium sulfonate, glutamic acid, tartaric acid, sodium tartrate, lactic acid, sodium lactate, fumaric acid, sodium fumarate, itaconic acid, ascorbic acid, sodium ascorbate, niacin, sodium niacin, fumaric acid, α-ketoglutarate, fruit acid, sodium fruit acid, acetic acid, oxalic acid, succinic acid, citric acid, sodium citrate, or any combination thereof.

In the preferred embodiment of the disclosure, the content of the grape powder is of 2-25% (w/w), the content of the grape seed extract is of 1-15% (w/w), and the content of arginine, L-arginine, vitamin C, vitamin E or any combination thereof is of 0.5-15% (w/w) in the composition.

In the preferred embodiment of the disclosure, the content of the grape powder is of 3-20% (w/w), the content of the grape seed extract is of 2-12% (w/w), the content of arginine, L-arginine, vitamin C, vitamin E or any combination thereof is of 1-12% (w/w), the content of maltodextrin is of 55-85% (w/w), and the content of the flavoring agents is of 0.1-5% (w/w) in the composition, wherein the flavoring agents are selected from citric acid, ethyl maltol, grape essence, sucralose, or any combination thereof.

In the preferred embodiment of the disclosure, the content of the grape powder is of 5-15% (w/w), the content of the grape seed extract is of 3-10% (w/w), the content of arginine, L-arginine, vitamin C, vitamin E or any combination thereof is of 2-10% (w/w), the content of maltodextrin is of 60-80% (w/w), and the content of the flavoring agents is of 0.5-4% (w/w) in the composition, wherein the flavoring agents are selected from citric acid, ethyl maltol, grape essence, sucralose, or any combination thereof.

In the preferred embodiment of the disclosure, the content of the grape powder is of 8-14% (w/w), the content of the grape seed extract is of 3-10% (w/w), the content of maltodextrin is of 65-80% (w/w), the content of arginine, L-arginine, vitamin C, vitamin E or any combination thereof is of 3-10% (w/w), and the content of flavoring agents is of 1-3% (w/w) in the composition, wherein the flavoring agents are selected from citric acid, ethyl maltol, grape essence, sucralose, or any combination thereof.

In the preferred embodiment of the disclosure, the content of the grape powder is of 10-20% (w/w), the content of the grape seed extract is of 1-10% (w/w), the content of maltodextrin is of 65-85% (w/w), the content of arginine and L-arginine is of 1-5% (w/w), the content of vitamin C is of 1-5% (w/w), and the content of the flavoring agents is 1-3% (w/w) in the composition, wherein the flavoring agents are selected from citric acid, ethyl maltol, grape essence, sucralose, or any combination thereof.

In the preferred embodiment of the disclosure, the composition optionally comprises lubricants, and the content of the lubricants in the composition is preferably of 0.5-5% (w/w), also preferably of 1-4% (w/w), more preferably of 2-3% (w/w).

In the preferred embodiment of the disclosure, the lubricants are selected from micro-powdered silica gel, magnesium stearate, talc powder, aluminum hydroxide, boric acid, hydrogenated vegetable oil, polyethylene glycol, or any combination thereof.

In the preferred embodiment of the disclosure, the composition optionally comprises donkey-hide gelatin.

In the preferred embodiment of the disclosure, the content of donkey-hide gelatin in the composition is of 2-12% (w/w), preferably of 2.5-10% (w/w), more preferably of 3-7% (w/w).

In the preferred embodiment of the disclosure, the content of the grape powder is of 14%, the content of the grape seed extract is of 3%, and the content of L-arginine, vitamin C, vitamin E or any combination thereof is of 2-4% in the composition.

In the preferred embodiment of the disclosure, the composition optionally comprises honeysuckle, platycodon grandiflorum, bitter almond, licorice, poria cocos, yam, dried tangerine peel, black plum, siraitia grosvenorii, lily, nicotinamide, or any combination thereof with a content of 8-25% (w/w), preferably of 10-20% (w/w), more preferably of 12-18% (w/w).

In the preferred embodiment of the disclosure, the preparation form of the composition is selected from any one of powder, tablet, capsule, granule, pill, pulvis, dropping pill, mixture, dew, effervescing agent, paste, emulsion and medicated tea.

Another object of the disclosure is to provide a preparation method of a pharmaceutical composition having efficacy of preventing and treating lung injury, the composition comprises a grape powder, a grape seed extract and a pharmaceutically acceptable carrier, the method comprises weighing and evenly mixing the necessary amount of the grape powder, the grape seed extract and the pharmaceutically acceptable carrier to obtain the composition.

In the preferred embodiment of the disclosure, the content of the grape powder is of 1-25% (w/w) and the content of the grape seed extract is of 0.5-15% (w/w) in the composition.

In the preferred embodiment of the disclosure, the content of the grape powder is of 3-20% (w/w) and the content of the grape seed extract is of 2-12% (w/w) in the composition.

In the preferred embodiment of the disclosure, the content of the grape powder is of 6-15% (w/w) and the content of the grape seed extract is of 3-10% (w/w) in the composition.

In the preferred embodiment of the present disclosure, the content of resveratrol in the grape powder is of 1-20% (w/w), preferably of 4-15% (w/w), more preferably of 5-8% (w/w).

In the preferred embodiment of the disclosure, the content of the proanthocyanidins in the grape seed extract is of 90-100% (w/w), preferably of 95-98% (w/w).

In the preferred embodiment of the disclosure, the proanthocyanidins are oligomeric proanthocyanidins.

In the preferred embodiment of the disclosure, the pharmaceutically acceptable carrier is selected from fillers, antioxidants, flavoring agents, or any combination thereof.

In the preferred embodiment of the disclosure, the content of the fillers in the composition is of 55-90% (w/w), preferably of 65-85% (w/w), more preferably of 70-80% (w/w).

In the preferred embodiment of the disclosure, the fillers are selected from maltodextrin, starch, lactose, sodium carboxymethyl starch, sodium starch glycolate, pregelatinized starch, modified starch, hydroxypropyl starch, corn starch, cellulose, microcrystalline cellulose, sodium carboxymethyl cellulose, ethyl cellulose, hydroxypropyl methylcellulose, calcium sulfate, calcium phosphate, calcium hydrogen phosphate, precipitated calcium carbonate, sorbitol, glycine, or any combination thereof.

In the preferred embodiment of the disclosure, the content of antioxidants in the composition is of 0.5-15% (w/w), preferably of 1-12% (w/w), more preferably of 2-10% (w/w).

In the preferred embodiment of the disclosure, the antioxidants are selected from arginine, L-arginine, vitamin C, vitamin E, tert-butyl hydroquinone, butyl hydroxyanisole, dibutyl hydroxytoluene, or any combination thereof.

In the preferred embodiment of the disclosure, the content of arginine, L-arginine, vitamin C, vitamin E or any combination thereof in the composition is of 0.5-15% (w/w), preferably of 1-12% (w/w), more preferably of 2-10% (w/w).

In the preferred embodiment of the disclosure, the content of the flavoring agents in the composition is of 0.1-5% (w/w), preferably of 0.2-4% (w/w).

In the preferred embodiment of the disclosure, the flavoring agents are selected from sucralose, grape essence, erythritol, xylitol, honey, sucrose, glucose, mogroside, malic acid, fumaric acid, citric acid, phosphoric acid, ethyl maltol, sodium citrate, sodium malate, acetic acid, sodium acetate, sodium hydrogen phosphate, sodium dihydrogen phosphate, carbonic acid, sodium carbonate, sulfonic acid, sodium sulfonate, glutamic acid, tartaric acid, sodium tartrate, lactic acid, sodium lactate, fumaric acid, sodium fumarate, itaconic acid, ascorbic acid, sodium ascorbate, niacin, sodium niacin, fumaric acid, α-ketoglutaric acid, fruit acid, sodium fruit acid, acetic acid, oxalic acid, succinic acid, citric acid, sodium citrate, or any combination thereof.

In the preferred embodiment of the present disclosure, the content of grape powder is of 2-25% (w/w), the content of grape seed extract is of 1-15% (w/w), and the content of arginine, L-arginine, vitamin C, vitamin E or any combination thereof is of 0.5-15% (w/w) in the composition.

In the preferred embodiment of the disclosure, the content of the grape powder is of 3-20% (w/w), the content of the grape seed extract is of 2-12% (w/w), the content of arginine, L-arginine, vitamin C, vitamin E or any combination thereof is of 1-12% (w/w), the content of maltodextrin is of 55-85% (w/w), and the content of the flavoring agents is of 0.1-5% (w/w) in the composition, wherein the flavoring agents are selected from citric acid, ethyl maltol, grape essence, sucralose, or any combination thereof.

In the preferred embodiment of the disclosure, the content of the grape powder is of 5-15% (w/w), the content of the grape seed extract is of 3-10% (w/w), the content of arginine, L-arginine, vitamin C, vitamin E or any combination thereof is of 2-10% (w/w), the content of maltodextrin is of 60-80% (w/w), and the content of the flavoring agents is of 0.5-4% (w/w) in the composition, wherein the flavoring agents are selected from citric acid, ethyl maltol, grape essence, sucralose, or any combination thereof.

In the preferred embodiment of the disclosure, the content of the grape powder is of 8-14% (w/w), the content of the grape seed extract is of 3-10% (w/w), the content of maltodextrin is of 65-80% (w/w), the content of arginine, L-arginine, vitamin C, vitamin E or any combination thereof is of 3-10% (w/w), and the content of the flavoring agents is of 1-3% (w/w) in the composition, wherein the flavoring agents are selected from citric acid, ethyl maltol, grape essence, sucralose, or any combination thereof.

In the preferred embodiment of the disclosure, the content of the grape powder is of 10-20% (w/w), the content of the grape seed extract is of 1-10% (w/w), the content of maltodextrin is of 65-85% (w/w), the content of arginine and L-arginine is of 1-5% (w/w), the content of vitamin C is of 1-5% (w/w), and the content of the flavoring agents is of 1-3% (w/w) in the composition, wherein the flavoring agents are selected from citric acid, ethyl maltol, grape essence, sucralose, or any combination thereof.

In the preferred embodiment of the disclosure, the composition optionally comprises lubricants, and the content of the lubricants in the composition is preferably of 0.5-5% (w/w), also preferably of 1-4% (w/w), more preferably of 2-3% (w/w).

In the preferred embodiment of the disclosure, the lubricants are selected from micro-powdered silica gel, magnesium stearate, talc powder, aluminum hydroxide, boric acid, hydrogenated vegetable oil, polyethylene glycol, or any combination thereof.

In the preferred embodiment of the disclosure, the composition optionally comprises donkey-hide gelatin.

In the preferred embodiment of the disclosure, the content of donkey-hide gelatin in the composition is of 2-12% (w/w), preferably of 2.5-10% (w/w), more preferably of 3-7% (w/w).

In the preferred embodiment of the disclosure, the content of the grape powder is of 14%, the content of grape seed extract is of 3%, and the content of L-arginine, vitamin C, vitamin E or any combination thereof is of 2-4% in the composition.

In the preferred embodiment of the disclosure, the composition optionally comprises honeysuckle, Platycodon grandiflorum, bitter almond, licorice, poria cocos, yam, dried tangerine peel, black plum, siraitia grosvenorii, lily, nicotinamide, or any combination thereof with a content of 8-25% (w/w), preferably of 10-20% (w/w), more preferably of 12-18% (w/w).

Another object of the disclosure is to provide a use of a pharmaceutical composition in the preparation of products for preventing and treating lung injury.

In the preferred embodiment of the disclosure, the lung injury is selected from any of acute lung injury, inhalation lung injury, smoke induced lung injury and PM2.5-induced lung injury, oxidative injury, ischemic lung injury, lung injury caused by virus infection, lung injury caused by ischemia and hypoxia, lung injury caused by hypertension, chronic obstructive pulmonary disease (COPD), or complications thereof.

The dosage of the composition of the disclosure is correlated with factors such as the patient's age, gender, health status, treatment status and combined medication, etc. The recommended dosage is of 5 g/time, 1-3 times/day.

The grape powder and the grape seed extract used in the disclosure are commercially available or prepared by conventional extraction methods in the art.

Unless otherwise stated, when the disclosure relates to the percentage between liquid and liquid, the percentage is volume/volume percentage; when the disclosure relates to the percentage between liquid and solid, the percentage is volume/weight percentage; when the disclosure relates to the percentage between solid and liquid, the percentage is weight/volume percentage; the rest is weight/weight percentage.

Compared with the prior art, the beneficial technical effects of the disclosure include:
1. The disclosure scientifically selects the components and proportions of the pharmaceutical composition, and the pharmaceutical composition has excellent efficacy of preventing and treating lung injury, oxidative injury, inhibiting pulmonary fibrosis and regulating lipid metabolism, significantly reduces lung injury, hypoxia injury and its adverse effect on lung function, significantly reduces PM2.5-induced lung histopathological changes and pulmonary inflammatory response, significantly improves PM2.5-induced breathing impairment, significantly improves the therapeutic efficacy of preventing and treating lung injury and antioxidation, and is conducive to the prevention and treatment of lung injury, lung injury caused by fine particles, lung injury caused by ischemia and hypoxia and other related diseases.
2. The preparation of the composition of the disclosure has the advantages of simple operation, green environmental protection, better cost, suitable for large-scale industrial production, and the like.

### BRIEF DESCRIPTION OF THE DRAWINGS

FIG. 1A-1C: FIG. 1A is the measured values of inspiratory time in the pulmonary function of hypertensive rats in each group, FIG. 1B is the measured values of expiratory time in the pulmonary function of hypertensive rats in each group, and FIG. 1C is the measured values of frequency of breathing in the pulmonary function of hypertensive rats in each group;

FIG. 2A-2C: FIG. 2A shows the blank pathological observation of hypertensive rats, FIG. 2B shows the pathological observation of model groups of hypertensive rats, and FIG. 2C shows the pathological observation of hypertensive rats using the composition of the disclosure;

FIG. 3 shows the measured values of percentage of monocytes of hypertensive rats in each group;

FIG. 4A-4F: FIG. 4A shows the measured values of LIX of hypertensive rats in each group, FIG. 4B shows the measured values of TNF-α of hypertensive rats in each group, FIG. 4C shows the measured values of IL-12p70 of hypertensive rats in each group, FIG. 4D shows the measured values of GRO/KC/CINC-1 of hypertensive rats in each group, FIG. 4E shows the measured values of IL-1α of hypertensive rats in each group, and FIG. 4F shows the measured values of IL-1β of hypertensive rats in each group;

FIG. 5A-5B: FIG. 5A shows the measured values of 8-OHdG in skeletal muscle of hypertensive rats in each group, and FIG. 5B shows the gene expression level of OGG1 of hypertensive rats in each group.

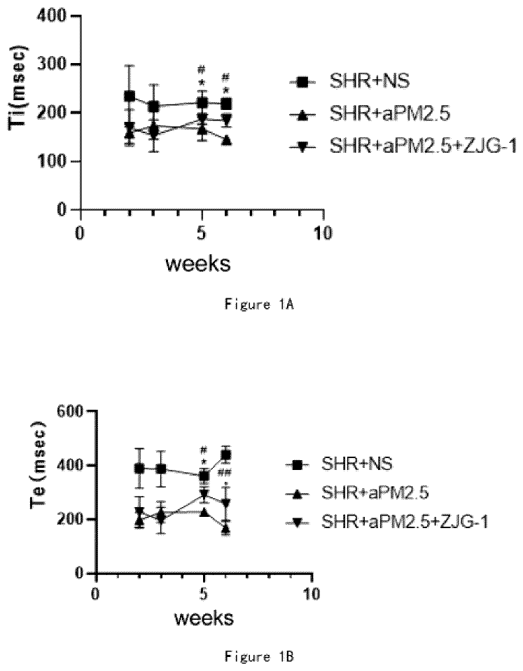

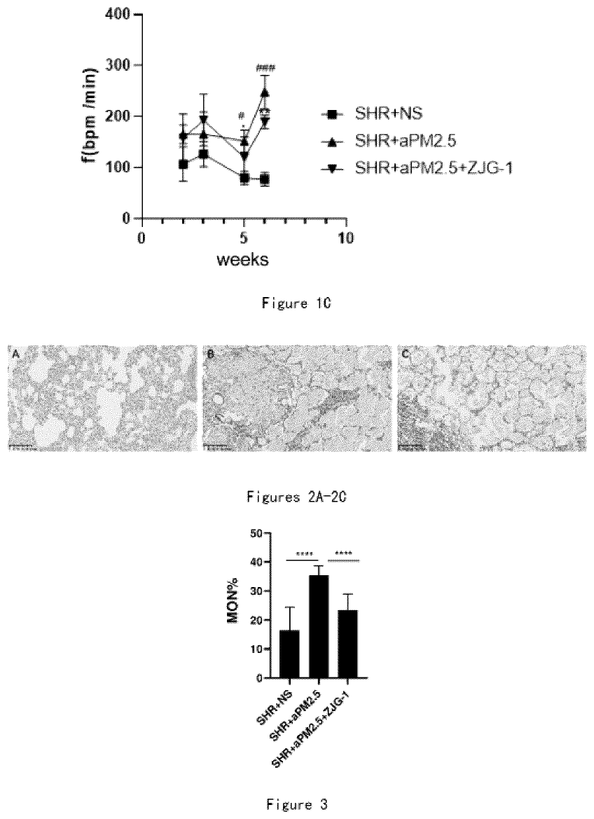

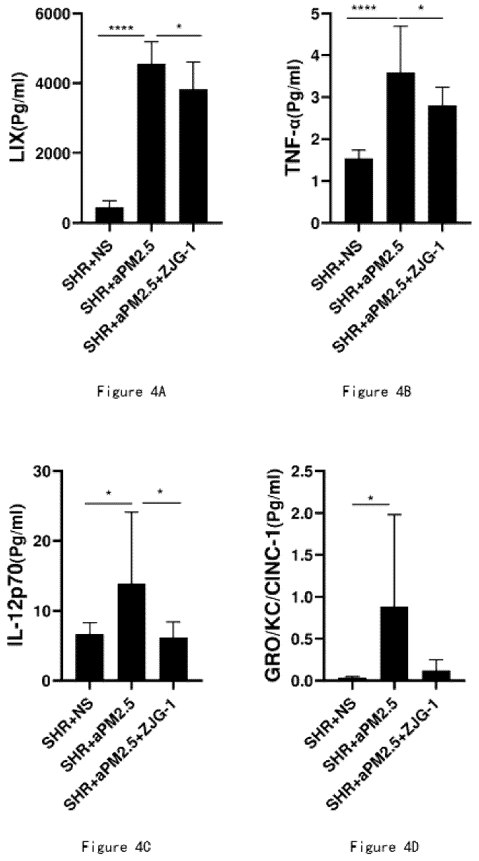

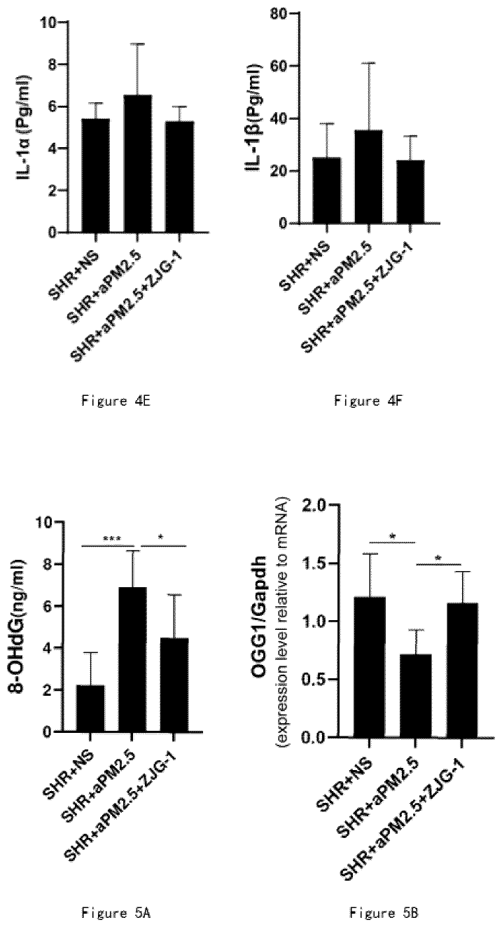

### DETAILED DESCRIPTION

The disclosure is described below with reference to examples. However, the disclosure is not limited to the examples.

The grape powder and the grape seed extract used in the specific examples are commercially available, in which the content of resveratrol in the grape powder is of 5% and the content of the proanthocyanidins in the grape seed extract is of 95%.

### Example 1 Preparation of the Composition of the Disclosure

20 g of a grape powder, 5 g of a grape seed extract, 5 g of L-arginine, 5 g of vitamin C and 65 g of maltodextrin are weighed and mixed evenly to obtain a composition.

### Example 2 Preparation of the Composition of the Disclosure

15 g of a grape powder, 10 g of a grape seed extract, 5 g of L-arginine, 5 g of vitamin C and 65 g of maltodextrin are weighed and mixed evenly to obtain a composition.

### Example 3 Preparation of the Composition of the Disclosure

20 g of a grape powder, 10 g of a grape seed extract, 2 g of L-arginine, 3 g of vitamin C and 65 g of corn starch are weighed and mixed evenly to obtain a composition.

### Example 4 Preparation of the Composition of the Disclosure

10 g of a grape powder, 5 g of a grape seed extract, 5 g of L-arginine, 5 g of vitamin C and 75 g of a corn starch are weighed and mixed evenly to obtain a composition.

### Example 5 Preparation of the Composition of the Disclosure

15 g of grape powder, 1 g of grape seed extract, 5 g of L-arginine, 4 g of vitamin E and 75 g of maltodextrin are weighed and mixed evenly to obtain a composition.

### Example 6 Preparation Example of the Composition of the Disclosure

75 g of maltodextrin, 10 g of a grape powder, 10 g of a grape seed extract, 2 g of L-arginine and 3 g of vitamin E are weighed and mixed evenly to obtain a composition.

### Example 7 Preparation of the Composition of the Disclosure

5 g of a grape powder, 10 g of a grape seed extract, 5 g of L-arginine, 5 g of vitamin C and 75 g of a resistant dextrin are weighed and mixed evenly to obtain a composition.

### Example 8 Preparation of the Composition of the Disclosure

5 g of a grape powder, 5 g of a grape seed extract, 2 g of L-arginine, 3 g of vitamin C and 85 g of a resistant dextrin are weighed and mixed evenly to obtain a composition.

### Example 9 Preparation of the Composition of the Disclosure

10 g of a grape powder, 1 g of a grape seed extract, 2 g of L-arginine, 2 g of vitamin C and 85 g of maltodextrin are weighed and mixed evenly to obtain a composition.

### Example 10 Preparation of the Composition of the Disclosure

12 g of a grape powder, 10 g of a grape seed extract, 5 g of L-arginine, 5 g of vitamin C, 65 g of maltodextrin, 1 g of citric acid, 1 g of ethyl maltol and 1 g of sucralose are weighed and mixed evenly to obtain a composition.

### Example 11 Preparation of the Composition of the Disclosure

10 g of a grape powder, 10 g of a grape seed extract, 1 g of L-arginine, 1 g of vitamin C, 75 g of maltodextrin, 1 g of citric acid, 1 g of ethyl maltol and 1 g of sucralose are weighed and mixed evenly to obtain a composition.

### Example 12 Preparation of the Composition of the Disclosure

15 g of a grape powder, 5 g of a grape seed extract, 1 g of L-arginine, 1 g of vitamin C, 75 g of maltodextrin, 1 g of citric acid, 1 g of ethyl maltol and 1 g of sucralose are weighed and mixed evenly to obtain a composition.

### Example 13 Preparation of the Composition of the Disclosure

10 g of a grape powder, 1 g of a grape seed extract, 1 g of L-arginine, 1 g of vitamin C, 85 g of maltodextrin, 1 g of citric acid, 0.5 g of ethyl maltol and 0.5 g of sucralose are weighed and mixed evenly to obtain a composition.

### Example 14 Preparation of the Composition of the Disclosure

10 g of a grape powder, 1 g of a grape seed extract, 1 g of L-arginine, 1 g of vitamin E, 85 g of maltodextrin, 1 g of citric acid, 0.5 g of ethyl maltol and 0.5 g of sucralose are weighed and mixed evenly to obtain a composition.

### Example 15 Preparation of the Composition of the Disclosure

14 g of a grape powder, 5 g of a grape seed extract, 2 g of L-arginine, 2 g of vitamin C and 77 g of maltodextrin are weighed and mixed evenly to obtain a composition.

### Example 16 Preparation of the Composition of the Disclosure

1 g of a grape powder, 15 g of a grape seed extract, 10 g of L-arginine, 10 g of vitamin C, 60 g of maltodextrin, 1 g of citric acid, 1 g of ethyl maltol and 2 g of sucralose are weighed and mixed evenly to obtain a composition.

### Example 17 Preparation of the Composition of the Disclosure

25 g of a grape powder, 0.5 g of a grape seed extract, 0.5 g of L-arginine, 0.5 g of vitamin C, 70 g of maltodextrin, 1 g of citric acid, 1 g of ethyl maltol and 1.5 g of sucralose are weighed and mixed evenly to obtain a composition.

### Example 18 Preparation of the Composition of the Disclosure

14 g of a grape powder, 3 g of a grape seed extract, 2 g of L-arginine, 2 g of vitamin C, 77.8 g of maltodextrin, 1 g of citric acid, 0.1 g of sucralose and 0.1 g of ethyl maltol are weighed and mixed evenly to obtain a composition.

### Example 19 Preparation of the Composition of the Disclosure

10 g log of a grape powder, 10 g of a grape seed extract, 1 g of L-arginine, 1 g of vitamin C, 75 g of maltodextrin, 1 g of citric acid, 1 g of ethyl maltol and 1 g of sucralose are weighed and mixed evenly to obtain a composition.

### Example 20 Preparation of the Composition of the Disclosure

10 g log of a grape powder, 10 g of a grape seed extract, 1 g of L-arginine, 1 g of vitamin C, 75 g of maltodextrin, 0.5 g of citric acid, 2 g of ethyl maltol and 0.5 g of sucralose are weighed and mixed evenly to obtain a composition.

### Example 21 Preparation of the Composition of the Disclosure

14 g of a grape powder, 5 g of a grape seed extract, 2 g of L-arginine, 2 g of vitamin C, 75 g of maltodextrin, 0.5 g of citric acid, 1 g of ethyl maltol and 0.5 g of sucralose are weighed and mixed evenly to obtain a composition.

### Example 22 Preparation of the Composition of the Disclosure

12 g of a grape powder, 8 g of a grape seed extract, 4 g of L-arginine, 4 g of vitamin C, 70 g of maltodextrin, 0.5 g of citric acid, 1 g of ethyl maltol and 0.5 g of sucralose are weighed and mixed evenly to obtain a composition.

### Example 23 Preparation of the Composition of the Disclosure

15 g of a grape powder, 5 g of a grape seed extract, 2 g of L-arginine, 2 g of vitamin C, 74 g of maltodextrin, 0.5 g of citric acid, 1 g of ethyl maltol and 0.5 g of sucralose are weighed and mixed evenly to obtain a composition.

### Example 24 Preparation of the Composition of the Disclosure

13 g of a grape powder, 6 g of a grape seed extract, 3 g of L-arginine, 3 g of vitamin C, 73.5 g of maltodextrin, 0.5 g of citric acid, 0.5 g of ethyl maltol and 0.5 g of sucralose are weighed and mixed evenly to obtain a composition.

### Example 25 Preparation of the Composition of the Disclosure

14 g of a grape powder, 5 g of a grape seed extract, 5 g of donkey-hide gelatin, 2 g of L-arginine, 2 g of vitamin C, 70 g of maltodextrin, 0.5 g of citric acid, 1 g of ethyl maltol and 0.5 g of sucralose are weighed and mixed evenly to obtain a composition.

### Test Example 1 Study on the Anti Lung Injury Efficacy of the Composition of the Disclosure

35 male SD rats of SPF grade with a weight range of 160-180 g were purchased from Beijing Vital River Laboratory Animal Technology Co., Ltd. The laboratory animal facilities maintain a barrier environment standard. The control range of main environmental indicators includes room temperature of 20-26° C., relative humidity of 40-70%, minimum ventilation rate of 15 times/hour, and lighting conditions with a light/dark=12 h:12 h. The animals were fed in polypropylene rat group boxes, with 3-5 rats in each box. The padding and cages are changed once or twice a week. During feeding, the animals are kept to be free to eat and move.

Artificial fine particles (aPM2.5) are provided by the Preparation Laboratory of Institute of Materia Medica, Chinese Academy of Medical Sciences.

Preparation of aPM2.5 suspension: take a 50 ml centrifuge tube, weigh 600 mg of artificial fine particles and dissolve them in 20 ml of normal saline to prepare aPM 2.5 suspension with a final concentration of 30 mg/ml. After mixing, ultrasonic crushing was carried out for 15 min under the condition of ice bath in a cell ultrasonic crusher with on-off intervals of 5 seconds each. After the preparation, the suspension was stored at −20° C. in a refrigerator and was used within one week. Before being used, it was melt in a water bath at 37° C., shaken and mixed evenly, and shaken well before being dripped.

aPM2.5 suspension poisoning method: aPM2.5 poisoning was performed by non-invasive tongue depressor intratracheal instillation. Before being dripped, aPM2.5 suspension was heated to 37° C. with a dripping volume of 1 ml/kg, and the rats were anesthetized with ether. The upper incisor teeth of the rats were hung on the cord at the upper end of the table top of the rat fixing table to make their bodies sag naturally. The rats were fixed on the table top in a supine position and the table top slopes 45 degrees for easy administration. The tongues of the rats were picked out with a gavage needle, the small lamp in front of a laryngoscope was turned on, and the plastic half funnel-shaped tip of the laryngoscope was inserted into their mouth to their throat to expose their throat, and then the glottic fissure with inverted “V” shape of throat thereof can be clearly observed. When it is opened, a small gavage needle was insert into the trachea and a liquid was slowly pushed to make the suspension of fine particles into the trachea. After dripping, the rats were removed from the fixed frame, immediately erected and rotated, and the lungs thereof were gently rubbed to make the particles evenly distributed in both lungs as much as possible. A blank control group was dripped with the same volume of normal saline. On the first day of the experiment, tracheal drip was administered for poisoning once every three days for a total of 12 times.

The rats were fasted but allowed access to water for 16 hours before dissection, and anesthetized with 2% pentobarbital sodium (dosage: 40 mg/kg) at 48 hours after the last poisoning, and then the rats were fixed on the operating table in a supine position.

The abdominal cavity was opened and blood was taken from the abdominal aorta; the chest cavity was opened and a bronchoalveolar lavage was carried out on the left lung to prepare bronchoalveolar lavage fluid (BALF); the middle lobe of right lung was removed and immersed in 4% of paraformaldehyde for fixation, which was used to prepare pathological sections; and after repacking, the remaining lung tissues were frozen in liquid nitrogen and transferred to −80° C. refrigerator for storage.

Put the grape powder, the grape seed extract and the composition of example 18 into a mortar respectively, grind them finely, add a small amount of 0.5% CMC-Na solution, grind and mix them evenly, and fix the volume to make a final concentration of 40 mg/ml. It is prepared every week and stored in a refrigerator at 4° C., and shake it well before use. Administer 1 ml/100 g medicine by gavage every day with a dosage of 400 mg/kg.

| | Tracheal drip | Tracheal drip | | Gavage dosage | Number of |
|---|---|---|---|---|---|
| Groups | reagents | dosage (mg/kg) | Gavage reagent | (mg/kg) | animals |
| Control | normal saline | 0 | 0.5% CMC-Na | 0 | 7 |
| group | | | | | |
| Model | aPM2.5 | 3 | 0.5% CMC-Na | 0 | 7 |
| group | Suspension | | | | |
| Group 1 | aPM2.5 | 3 | grape powder | 400 | 7 |
| | Suspension | | | | |
| Group 2 | aPM2.5 | 3 | grape seed | 400 | 7 |
| | Suspension | | extract | | |
| Group 3 | aPM2.5 | 3 | composition of | 400 | 7 |
| | Suspension | | example 18 | | |

Control group (C): 0.1 ml/100 g normal saline was administered by tracheal drip once every three days, and 0.5% CMC-Na was administered by gavage every day. Each drip was carried out in the afternoon after gavage, and the rats were killed on the 37th day.

Model group (M): 0.1 ml/100 g (30 mg/ml) suspension of fine particles was administered by tracheal drip once every three days, and 0.5% CMC-Na was administered by gavage every day. Each drip was carried out in the afternoon after gavage, and the rats were killed on the 37th day.

Group 1 (Grape Skin Extract): 0.1 ml/100 g (30 mg/ml) suspension of fine particles was administered by tracheal drip once every three days, and 1 ml/100 g (400 mg/kg) grape powder suspension was administered by gavage every day. Each drip was carried out in the afternoon after gavage, and the rats were killed on the 37th day.

Group 2 (Grape Seed Extract): 0.1 ml/100 g (30 mg/ml) suspension of fine particles was administered by tracheal drip once every three days, and 1 ml/100 g (400 mg/kg) grape seed extract suspension was administered by gavage every day. Each drip was carried out in the afternoon after gavage, and the rats were killed on the 37th day.

Group 3 (composition of example 18): 0.1 ml/100 g (30 mg/ml) of suspension of fine particles was administered by tracheal drip once every three days, and 1 ml/100 g (400 mg/kg) suspension of composition of example 18 was administered by gavage every day. Each drip was carried out in the afternoon after gavage, and the rats were killed on the 37th day.

During the experiments, pulmonary function indexes including Inspiratory Time (Ti), Peak Expiratory Flow (PIF), Minute Ventilation Volume (MV) and frequency of breathing (fbpm) were measured before administration, on the 11th, 23rd and 35th days of modeling, respectively, and the differential leukocyte count, inflammatory factors and oxidative damage in the gavage fluid of lung cell were detected.

The data were processed by Excel 2016 software, and the results were expressed by mean±standard deviation (X±SD). The homogeneity of variance between the groups was tested by FTEST, and then the significance of the difference between the two groups was compared by bilateral Student's T-Test, and P<0.05 was the statistical difference standard. The results are shown in tables 1-4.

TABLE 1
Study on protective effect of lung function injury in rats with aPM2.5 poisoning

| | | | Peak Expiratory | Minute Ventilation | Frequency of |
|---|---|---|---|---|---|
| | | Inspiratory Time | Flow | Volume | Breathing |
| Groups | | Ti, msec | PIF, ml/s | MV, ml | F, bpm |
| D0 | C | 234.76 ± 14.27 | 6.19 ± 1.04 | 103.00 ± 8.36 | 110.61 ± 7.41 |
| | M | 227.04 ± 26.04 | 6.12 ± 1.39 | 102.47 ± 16.41 | 113.56 ± 12.82 |
| | G1 | 233.71 ± 24.44 | 5.92 ± 0.60 | 100.67 ± 7.75 | 110.71 ± 15.72 |
| | G2 | 239.09 ± 21.00 | 5.85 ± 0.74 | 102.73 ± 13.37 | 111.30 ± 9.47 |
| | G3 | 239.74 ± 25.91 | 5.67 ± 0.92 | 95.23 ± 11.69 | 106.00 ± 10.87 |
| D11 | C | 262.76 ± 22.64 | 6.00 ± 0.75 | 102.23 ± 11.52 | 100.87 ± 8.74 |
| | M | 197.43 ± 35.00** | 7.34 ± 1.11* | 121.87 ± 13.24* | 124.26 ± 9.15** |
| | G1 | 210.34 ± 23.66 | 6.27 ± 0.62^{#} | 106.80 ± 6.45^{#} | 115.38 ± 3.44 |
| | G2 | 198.82 ± 15.63 | 6.53 ± 0.72 | 111.51 ± 8.85 | 124.73 ± 6.68 |
| | G3 | 224.52 ± 9.17^{※※} | 6.24 ± 0.40^{#} | 104.62 ± 8.79^{#} | 112.80 ± 2.82^{##, ※※} |
| D23 | C | 282.43 ± 16.07 | 7.14 ± 0.43 | 117.75 ± 4.50 | 88.83 ± 4.17 |
| | M | 197.56 ± 23.49** | 9.26 ± 0.83** | 154.26 ± 18.90** | 120.62 ± 12.83** |
| | G1 | 227.88 ± 17.22^{#} | 7.94 ± 0.90^{#} | 135.19 ± 8.21^{#} | 106.63 ± 9.66^{#} |
| | G2 | 224.60 ± 30.55 | 8.42 ± 0.89 | 135.16 ± 9.22# | 110.31 ± 5.09 |
| | G3 | 240.81 ± 6.79^{##} | 7.48 ± 0.72^{##} | 125.83 ± 6.18^{##, ※, Δ} | 96.35 ± 5.92^{##, ※, ΔΔ} |
| D35 | C | 282.10 ± 25.23 | 7.89 ± 0.78 | 130.11 ± 6.16 | 88.69 ± 3.80 |
| | M | 214.64 ± 29.15** | 9.71 ± 1.02** | 168.18 ± 11.26** | 117.05 ± 11.56** |
| | G1 | 241.11 ± 7.77 | 8.49 ± 0.78^{#} | 147.23 ± 9.53^{##} | 104.20 ± 10.01^{#} |
| | G2 | 232.28 ± 27.32 | 8.38 ± 0.98^{#} | 150.73 ± 11.46^{#} | 105.15 ± 10.39 |
| | G3 | 251.22 ± 21.38^{#} | 8.15 ± 0.71^{##} | 134.27 ± 10.09^{##, ※, Δ} | 96.73 ± 5.56^{##} |

Note:
compared with the control group,
*P < 0.05,
**P < 0.01;
compared with the model group,
^{#}P < 0.05,
^{##}P < 0.01;
compared with grape powder group (G1),
^{※}P < 0.05,
^{※※}P < 0.01;
and compared with grape seed extract (G2),
^{Δ}P < 0.05,
^{ΔΔ}P < 0.01

TABLE 2
Effect on white blood cells and classification proportion of BALF
lavage liquid in lung injury model of rats with aPM tracheal drip

| Groups | WBC(10⁹/L) | LYM % | MON % | NEUT % | EOS % | BAS % |
|---|---|---|---|---|---|---|
| C | 33.2 ± 10.5 | 13.9 ± 2.5 | 18.1 ± 3.7 | 33.2 ± 7.5 | 10.5 ± 2.4 | 24.2 ± 8.5 |
| M | 89.1 ± 20.4** | 18.2 ± 6.0 | 18.0 ± 5.2 | 42.7 ± 6.8* | 11.0 ± 2.2 | 10.1 ± 2.4** |
| G1 | 53.7 ± 32.5^{#} | 11.2 ± 4.9^{#} | 19.9 ± 11.2 | 43.9 ± 15.0 | 10.5 ± 3.8 | 14.5 ± 5.0 |
| G2 | 59.0 ± 45.4 | 15.5 ± 3.0 | 27.0 ± 8.6^{#} | 29.5 ± 7.3^{##} | 9.4 ± 4.4 | 18.6 ± 4.7^{##} |
| G3 | 47.4 ± 14.4^{##} | 16.1 ± 2.8^{※} | 21.8 ± 9.8 | 30.3 ± 5.8^{##} | 13.0 ± 5.6 | 19.7 ± 3.8^{##, ※} |

Note:
compared with the control group,
*P < 0.05,
**P < 0.01;
compared with the model group,
^{#}P < 0.05,
^{##}P < 0.01;
compared with grape powder group (G1),
^{※}P < 0.05.
WBC: white blood cell count;
LYM %: percentage of lymphocytes;
MON %: percentage of monocytes;
NEUT %: percentage of neutrophils;
EOS %: percentage of eosinophils;
BAS %: percentage of basophils.

TABLE 3
Comparison of effect on cytokines of BALF lavage fluid
in rats with lung function injury caused by aPM

| Group | TNF-α (pg/ml) | IL-10 (pg/ml) |
|---|---|---|
| C | 89.7 ± 15.6 | 19.0 ± 0.7 |
| M | 121.2 ± 18.0** | 14.1 ± 2.0** |
| G1 | 98.1 ± 12.0# | 16.1 ± 2.4 |
| G2 | 106.3 ± 6.7 | 15.5 ± 2.8 |
| G3 | 94.9 ± 12.6## | 17.3 ± 1.1# |

Note:
compared with the control group,
**P < 0.01;
compared with the model group,
#P < 0.05,
##P < 0.01
TNF-α: Tumor necrosis factor;
IL-10: interleukin-10.

TABLE 4
Effect on oxidation indexes in lung tissue of
rats with lung function injury caused by aPM

| Groups | SOD (U/mgprot) | MDA (nmol/mgprot) |
|---|---|---|
| C | 61.6 ± 7.4 | 0.49 ± 0.12 |
| M | 49.1 ± 3.5** | 0.74 ± 0.13** |
| G1 | 54.0 ± 3.8# | 0.59 ± 0.08# |
| G2 | 50.4 ± 5.2 | 0.64 ± 0.04 |
| G3 | 58.9 ± 7.2## | 0.52 ± 0.03##, Δ |

Note:
compared with the control group,
**P < 0.01;
compared with the model group,
#P < 0.05,
##P < 0.01;
compared with grape seed extract group (G2),
ΔP < 0.05
Sod: superoxide dismutase;
MDA: malondialdehyde
G1-G3 can improve lung function, reduce elevated level of white blood cells and TNF-α level in BALF of model rats, increase IL-10 and SOD in lung tissue, and reduce MDA in lung tissue.
G3 has the strongest effect on improving lung function in rats.

### Test Example 2 Study on Protective Effect of the Composition of the Disclosure on Lung Injury in Hypertensive Rat Model

The test method of test example 2 is the same as that of test example, including the provision of artificial fine particles (aPM2.5) and preparation of aPM2.5 suspension, aPM2.5 suspension poisoning method, preparation of the composition and administration method of the composition of example 18, etc.

30 rats aged 6 weeks with primary hypertensive SHR were used as experimental animals and divided into three groups: control group (SHR+NS), model group (SHR+PM2.5) and drug group (SHR+PM2.5+drug).

The rats were fed adaptively for one week after purchasing, and the blood pressure and pulmonary function were measured, then prophylactic administration is carried out and the dosage was of 1 g/kg. One week after administration, 100 mg/kg aPM2.5 suspension were administered to rats by tracheal drip once a week until the results of non-invasive pulmonary function showed that the pulmonary function of the model group decreased significantly. Then the experiment was ended.

An EMKA pulmonary function monitoring system is used to monitor the whole respiratory state. Compared with the model group, the drug group can effectively improve Ti and Te and reduce the frequency of breathing, which has a better protective effect on pulmonary function, and significantly improves the appearance of granuloma, chronic inflammation and exudation in lungs of rats in the model group, wherein granuloma is diffusely distributed lesions characterized by fibrous tissue hyperplasia in the form of nodules. It significantly reduces the level of pro-inflammatory related cytokines and the level of 8-OHdG in skeletal muscle, significantly improves skeletal muscle atrophy caused by lung function decline, and significantly improves the repair of DNA damage.

The above description of the particular embodiments of the disclosure is not intended to limit the disclosure. Those skilled in the art can make various modifications or variations according to the disclosure, provided that they do not deviate from the spirit of the disclosure, they should belong to the protection scope of the claims of the disclosure.

What is claimed is:

1. A pharmaceutical composition having the efficacy of preventing and treating lung injury, wherein the composition comprises a grape powder, a grape seed extract and a pharmaceutically acceptable carrier, the content of the grape powder is of 1-25% w/w and the content of the grape seed extract is of 0.5-15% w/w in the composition.

2. The composition according to claim 1, wherein the content of resveratrol in the grape powder is of 1-20% w/w.

3. The composition according to claim 1, wherein the content of proanthocyanidins in the grape seed extract is of 90-100% w/w.

4. The composition according to claim 3, wherein the proanthocyanidins are oligomeric proanthocyanidins.

5. The composition according to claim 1, wherein the pharmaceutically acceptable carrier is selected from fillers, antioxidants, flavoring agents, or any combination thereof.

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. The composition according to claim 5, wherein the content of the antioxidants in the composition is of 0.5-15% w/w, the antioxidants are selected from arginine, L-arginine, vitamin C, vitamin E, tert-butylhydroquinone, butyl hydroxyanisole, dibutyl hydroxytoluene, ascorbic acid, sodium ascorbate, or any combination thereof, and the content of arginine, L-arginine, vitamin C, vitamin E or any combination thereof in the composition is of 0.5-15% w/w.

22. The composition according to claim 21, wherein the content of the grape powder is of 2-25% w/w, the content of the grape seed extract is of 1-15% w/w, and the content of arginine, L-arginine, vitamin C, vitamin E or any combination thereof is of 0.5-15% w/w in the composition.

23. The composition according to claim 21, wherein the content of the grape powder is of 14% w/w, the content of the grape seed extract is of 3% w/w, and the content of L-arginine, vitamin C, vitamin E and any combination thereof is of 2-4% w/w in the composition.

24. The composition according to claim 21, wherein the content of the fillers in the composition is of 55-90% w/w, the fillers are selected from maltodextrin, starch, lactose, sodium carboxymethyl starch, sodium starch glycolate, pregelatinized starch, modified starch, hydroxypropyl starch, corn starch, cellulose, microcrystalline cellulose, sodium carboxymethyl cellulose, ethyl cellulose, hydroxypropyl methylcellulose, calcium sulfate, calcium phosphate, calcium hydrogen phosphate, precipitated calcium carbonate, sorbitol, glycine, or any combination thereof.

25. The composition according to claim 24, wherein the content of the flavoring agents in the composition is of 0.1-5% w/w, and the flavoring agents are selected from sucralose, grape essence, erythritol, xylitol, honey, sucrose, glucose, mogroside, malic acid, fumaric acid, citric acid, phosphoric acid, ethyl maltol, sodium citrate, sodium malate, acetic acid, sodium acetate, sodium hydrogen phosphate, sodium dihydrogen phosphate, carbonic acid, sodium carbonate, sulfonic acid, sodium sulfonate, glutamic acid, tartaric acid, sodium tartrate, lactic acid, sodium lactate, sodium fumarate, itaconic acid, niacin, sodium nicotinate, α-ketoglutaric acid, fruit acid, sodium fruit acid, oxalic acid, succinic acid, or any combination thereof.

26. The composition according to claim 25, wherein the content of the grape powder is of 3-20% w/w, the content of the grape seed extract is of 2-12% w/w, the content of arginine, L-arginine, vitamin C, vitamin E or any combination thereof is of 1-12% w/w, the content of maltodextrin is of 55-85% w/w, and the content of the flavoring agents is of 0.1-5% w/w in the composition, wherein the flavoring agents are selected from citric acid, ethyl maltol, grape essence, sucralose, or any combination thereof.

27. The composition according to claim 26, wherein the content of the grape powder is of 5-15% w/w, the content of the grape seed extract is of 3-10% w/w, the content of arginine, L-arginine, vitamin C, vitamin E or any combination thereof is of 2-10% w/w, the content of maltodextrin is of 60-80% w/w, and the content of the flavoring agents is 0.5-4% w/w in the composition, wherein the flavoring agents are selected from citric acid, ethyl maltol, grape essence, sucralose or any combination thereof.

28. The composition according to claim 25, wherein the content of the grape powder is of 8-14% w/w, the content of the grape seed extract is of 3-10% w/w, the content of maltodextrin is of 65-80% w/w, the content of arginine, L-arginine, vitamin C, vitamin E or any combination thereof is of 3-10% w/w, and the content of the flavoring agents is of 1-3% w/w in the composition, wherein the flavoring agents are selected from citric acid, ethyl maltol, grape essence, sucralose or any combination thereof.

29. The composition according to claim 25, wherein the content of the grape powder is of 10-20% w/w, the content of the grape seed extract is of 1-10% w/w, the content of maltodextrin is of 65-85% w/w, the content of L-arginine is of 1-5% w/w, the content of vitamin C is of 1-5% w/w, and the content of the flavoring agents is of 1-3% w/w in the composition, wherein the flavoring agents are selected from citric acid, ethyl maltol, grape essence, sucralose or any combination thereof.

30. The composition according to claim 1, wherein the composition comprises lubricants, and the lubricants are selected from micro-powdered silica gel, magnesium stearate, talc powder, aluminum hydroxide, boric acid, hydrogenated vegetable oil, polyethylene glycol, or any combination thereof.

31. The composition according to claim 1, wherein the composition comprises donkey-hide gelatin, and the content of donkey-hide gelatin in the composition is of 2-12% w/w.

32. The composition according to claim 1, wherein the composition comprises honeysuckle, platycodon grandiflorum, bitter almond, licorice, poria cocos, yam, dried tangerine peel, black plum, siraitia grosvenorii, lily, nicotinamide or any combination thereof with a content of 8-25% w/w.

33. The composition according to claim 1, wherein the preparation form of the composition is selected from any one of powder, tablet, capsule, granule, pill, pulvis, dropping pill, mixture, dew, effervescing agent, paste, emulsion and medicated tea.

34. A preparation method of the pharmaceutical composition having the efficacy of preventing and treating lung injury according to claim 1, wherein the composition comprises a grape powder, a grape seed extract and a pharmaceutically acceptable carrier, the method comprises weighing and evenly mixing the necessary amount of the grape powder, the grape seed extract and the pharmaceutically acceptable carrier to obtain the composition.

35. A method for preventing and treating lung injury with the pharmaceutical composition having the efficacy of preventing and treating lung injury according to claim 1, the method comprises administering the pharmaceutical composition in the necessary amount to a subject.
